(19) United States
(12) Patent Application Publication
(10) Pub. No.: US 2023/0127624 A1
(43) Pub. Date: Apr. 27, 2023

(54) TRANSNASAL AND TRANSORAL NASOPHARYNGEAL DIAGNOSTICS AND THERAPEUTICS

(71) Applicant: BHL Patent Holdings LLC, Philadelphia, PA (US)

(72) Inventor: Bruce H Levin, Philadelphia, PA (US)

(21) Appl. No.: 17/975,144

(22) Filed: Oct. 27, 2022

Related U.S. Application Data
(60) Provisional application No. 63/263,144, filed on Oct. 27, 2021.
(60) Provisional application No. 63/268,717, filed on Mar. 1, 2022.

(51) Int. Cl. A61B 10/02; A61M 31/00

(52) U.S. Cl. CPC A61B 10/02; A61M 31/00; A61M 2210/1046

(57) ABSTRACT

Disclosed herein are devices and methods for improved transnasal and transoral nasopharyngeal diagnostics and therapeutics, particularly to detect the presence of infectious agents such as a coronavirus, for example SARS-CoV-2, responsible for COVID-19, and other viral agents.

This application claims priority from Provisional Application No. 63/268,717, filed Mar. 1, 2022 and Provisional Application No. 63/263,144 filed Oct. 27, 2021, the disclosures of each of which are hereby incorporated herein by reference in their entireties.

### FIELD

The invention most generally relates to transnasal and transoral nasopharyngeal diagnostics and therapeutics, particularly to detect the presence of infectious agents such as coronaviruses, including SARS-CoV-2, and other viral agents.

### BACKGROUND

Currently available testing for coronaviruses, including SARS-CoV-2, responsible for COVID-19, is suboptimal for multiple reasons. Current methodologies involve transnasal or transoral introduction of a straight mounted absorbent tip either through one or both nares or through the mouth. The transnasal or intranares approach is uncomfortable, and potentially traumatic, necessitating a smaller diameter sampler size. The probe used for such an analysis has an essentially generally unidimensional and curvilinear or oval single surface contact sampling surface.

This may have ramifications regarding attempts at nasopharyngeal sampling because of the presence of conchae, cysts, mucosal solid build up, or a deviated septum or other anatomic variance and potential pathologies. The sampling is generally done quickly because of the discomfort, and often times the nasopharynx is not reached, and even when contact is made the necessary care to avoid pharyngeal trauma often results in inadequate sampling.

The inventor notes several individuals who certainly had significant Covid symptoms and signs, including typical chest x-ray or other findings, and who nonetheless tested negative via nasal sample. These false negatives were generally attributed to the presence of early stage of the disease wherein the viral load was not high enough to be sampled. However the inventor notes that at least in a few of these cases the disease was progressive, and subsequent retesting was positive. Therefore, the test may have not been accurately performed, or the current prevalent method required a higher than optimal viral particle load to be noted as positive on these tests.

The inventor himself noted that during a covid test, the swab was held up intranasally and did not contact the nasopharynx, until this was corrected quite uncomfortably. Further, self-testing at home, or using a mail-in test kit, has been noted by many people to be uncomfortable, and is accompanied by a significant likelihood of inadequate sampling. Therefore, the likelihood of false negatives among self-testing individuals is significant.

There remains a need for improved diagnostic devices and methods directed to coronavirus infections, including COVID-19, and other viral infections of the upper respiratory tract.

### BRIEF DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

In some embodiments of any of the compositions or methods described herein, a range is intended to comprise every integer or fraction or value within the range.

Embodiments described herein as “comprising” one or more features may also be considered as disclosure of the corresponding embodiments “consisting of” and/or “consisting essentially of” such features.

Disclosed herein are methods and devices for testing for the presence of viruses, including the SARS-CoV-2 Covid virus, using suitably designed transnasal or transoral probes. In some embodiments, the probe positions a sampling tip in proximity to the nasal pharynx.

Accordingly, provided herein is a device for contacting the nasal pharynx via either the nasal cavity or the oral cavity, the device comprising:
a rodlike shaft for manual manipulation; and
one or more sampling tips at the distal end;
wherein the device has an upward curve or bend, providing access to the nasal pharynx.

In some embodiments, the one or more sampling tips comprise absorbent material. In some embodiments, the one or more sampling tips are substantially spherical in shape. In some embodiments, the one or more sampling tips are substantially ellipsoidal in shape. In some embodiments, the one or more sampling tips have the shape of a prolate ellipsoid. In some embodiments, the one or more sampling tips have the shape of an oblate ellipsoid. In some embodiments, the one or more sampling tips are substantially cylindrical in shape.

In some embodiments, the one or more sampling tips each contains a segmented sampling tip, each segment of which comprises an individual piece of absorbent material. In further embodiments, each individual piece of absorbent material can absorb a quantity of medicament. In further embodiments, each individual piece of absorbent material can be brought into contact with the mucosa by rotation around one or more axes of the device, thereby providing sequential dosage to the mucosa.

In some embodiments, each of the one or more sampling tips is connected to the rodlike shaft via a single point of attachment oriented on one end of its symmetry axis. In some embodiments, each of the one or more sampling tips is connected to the rodlike shaft via two points of attachment oriented on opposite ends of its symmetry axis.

In some embodiments, the one or more sampling tips can freely rotate on an axis. In some embodiments, the axis of rotation is substantially parallel to the shaft at the distal end of the device.

In some embodiments, the device is extensible. In some embodiments, the shaft proximal to the upward curve or bend is extensible. In some embodiments, the shaft distal to the upward curve or bend is extensible.

In some embodiments, the device further comprises one or more reservoirs in proximity to the one or more sampling tips. In some embodiments, the rotation of a sampling tip on an axis causes distribution of material within a reservoir onto the surface of the tip.

In some embodiments, the device further comprises a mechanism for delivering a liquid from the one or more reservoirs to the tip. The mechanism may be chosen from a squeeze bulb, a plunger, and a pressurized container. In some embodiments, the mechanism can be manipulated so as to deliver a single aliquot to the tip. In some embodiments, the volume of the single aliquot is fixed. In some embodiments, the volume of the single aliquot is adjustable. In some embodiments, the mechanism can be sequentially manipulated so as to deliver a plurality of individual aliquots to the tip. In some embodiments, the volume of the individual aliquot is fixed. In some embodiments, the volume of the individual aliquot is adjustable.

In some embodiments, the device further comprises one or more reservoirs in the proximal end. In some further embodiments, the device further comprises one or more channels within the shaft to convey contents of the one or more reservoirs to the sampling tips. In some further embodiments, each of the one or more channels connects with an aperture in one of the one or more sampling tips.

Also provided herein are methods for detecting or quantifying a microbe in the upper respiratory tract, the method comprising the steps of:
transnasally or transorally inserting a device as disclosed herein;
contacting the tip of the device with the nasal pharynx;
manipulating the device so as to harvest material from the nasal pharynx; and withdrawing the device.

In some embodiments, the microbe is a virus. In some embodiments, the microbe is a coronavirus. In some embodiments, the virus is chosen from a rhinoviruses, an influenza viruses, and a parainfluenza virus. In some embodiments, the virus is SARS-CoV-2.

In some embodiments, the microbe is a bacterium. In some embodiments, the bacterium is chosen from Streptococcus pneumoniae, Haemophilus influenzae, Moraxella catarrhalis, and Streptococcus pyogenes.

Also disclosed herein are methods and devices for treating illnesses of the upper respiratory tract, and/or alleviating the symptoms of nasal illnesses.

Accordingly, also provided herein are methods for treating or ameliorating an ailment of the upper respiratory tract; the method comprising the steps of:
transnasally or transorally inserting a device as disclosed herein;
contacting the tip of the device with the nasal pharynx;
manipulating the device so as to apply medicament contained in the one or more reservoirs onto the surface of nasal pharynx; and
withdrawing the device.

In some embodiments, the step of contacting the tip of the device with the nasal pharynx is performed prior to the step of manipulating the device.

In some embodiments, the step of manipulating the device consists of rotating the proximal end of the shaft of the device. In some embodiments, rotation of the shaft causes the tip to sweep an arc on the surface of the nasal pharynx. In some embodiments, rotation of the shaft causes the tip to contact a larger area of the nasal pharynx than in the absence of rotation.

In some embodiments, the medicament for application onto the surface of the nasal pharynx is contained within one or more reservoirs in the device. In some embodiments, application of the medicament is performed with a mechanism for delivering the medicament from the one or more reservoirs to the tip. The mechanism may be chosen from a squeeze bulb, a plunger, and a pressurized container. In some embodiments, the mechanism provides a single aliquot of medicament to the tip. In some embodiments, the mechanism provides a plurality of individual aliquots to the tip.

In some embodiments, medicament is applied in essentially a single dose. In some embodiments, medicament is applied over a period of time. In some embodiments, application of medicament over a period of time is accomplished by contacting an absorbent tip with the mucosa, the absorbent tip containing medicament. In some embodiments, a plurality of aliquots of medicament is applied over a period of time. In some embodiments, the plurality of aliquots is provided by a segmented sampling tip, each segment of which comprises an individual piece of absorbent material. In some embodiments, the plurality of aliquots is provided by periodic delivery of aliquots from a reservoir within the device.

In some embodiments, the device comprises a moveable part at the tip. In some embodiments, the moveable part is a roller. In some embodiments, the roller can freely rotate about an axis of rotation that is substantially parallel to the shaft at the distal end of the device. In some embodiments, rotation of the proximal end of the shaft produces a rolling motion of the roller about the axis of rotation. In some embodiments, the rolling motion of the roller acts to maintain continuous contact between the roller and the nasal pharynx. In some embodiments, the continuous contact between the tip and the nasal pharynx is maintained without significant displacement of the surface of the roller relative to the nasal pharynx.

In some embodiments, the device comprises an expandable part at the tip. In some embodiments, the expandable part can be inflated and/or deflated. In some embodiments, an inflation and/or deflation operation can be controlled by a mechanism at the proximal end of the device. In some embodiments, the method further comprises the step of expanding the expandable tip subsequent to insertion of the device in proximity to the nasal pharynx, thereby bringing the exterior of the expandable tip into contact with the nasal pharynx. In some embodiments, the method further comprises the step of contracting the expandable tip prior to withdrawal of the device.

In some embodiments, the medicament is an antiviral. In some embodiments, the antiviral is chosen from remdesivir, galidesivir, favilavir/avifavir, mulnupiravir (MK-4482/EIDD 2801), AT-527, AT-301, BLD-2660, favipiravir, camostat, SLV213 emtrictabine/tenofivir, clevudine, dalcetrapib, boceprevir and ABX464, glucocorticoids such as dexamethasone and hydrocortisone, convalescent plasma, a recombinant human plasma such as gelsolin (Rhu-p65N), monoclonal antibodies such as regdanvimab (Regkirova), ravulizumab (Ultomiris), VIR-7831/VIR-7832, BRI 1-196/BRI I-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Maris), gimsilumab and otilimab, antibody cocktails such as casirivimab/imdevimab (REGN-Cov2), recombinant fusion protein such as MK-7110 (CD24Fc/SACCOVID), anticoagulants such as heparin and apixaban, IL-6 receptor agonists such as tocilizumab (Actemra) and sarilumab (Kevzara), PlKfyve inhibitors such as apilimod dimesylate, RIPK1 inhibitors such as DNL758, VIP receptor agonists such as PB1046, SGLT2 inhibitors such as dapaglifozin, TYK inhibitors such as abivertinib, kinase inhibitors such as ATR-002, bemcentinib, acalabrutinib, baricitinib and losmapimod, H2 blockers such as famotidine, anthelmintics such as niclosamide, furin inhibitors such as diminazene.

In some embodiments, the medicament is an antibacterial. In some embodiments, the antibacterial is chosen from a penicillin, a cephalosporin, vancomycin, rifamycin, rifampin, teicoplanin, sulfacetamide, amoxicillin, novobiocin, a tetracycline compound, tetracycline, oxytetracycline, methacycline, minocycline, chlortetracycline, doxycycline, hydropyracycline, demecycline, sulfanilamide, sulfamethoxazole, norfloxacin, gatifloxacin, gemifloxacin, an antitubercular compound, isoniazid, rifampin, streptomycin, ciprofloxacin, moxifloxacin, and aminosalicylic acid.

### BRIEF DESCRIPTION OF THE DRAWINGS

The foregoing summary, as well as the following detailed description of the presently disclosed technology, will be better understood when read in conjunction with the appended drawings, wherein like numerals designate like elements throughout. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 depicts (a) the region around the nasal cavity: nasopharynx 11 roof of the mouth 12 turbinates 13 skull bone 14 superior nasal cartilage 15 nasal cartilage 16 air flow 17 (b) orientation of a straight probe inserted through a naris: upward orientation 18 horizontal orientation 19.

FIG. 2 is adapted from representative images of an oropharyngeal swab robot and it sampling processes. (Shao-Qiang Li et al. Clinical application of an intelligent oropharyngeal swab robot: implication for the COVID-19 pandemic. European Respiratory Journal 2020 56: 2001912; DOI: 10.1183/13993003.01912-2020).

FIG. 3 depicts a procedure for transoral approach of a sampling tip for testing, treatment, and/or amelioration of symptoms.

FIG. 4 depicts devices and procedures for transoral approach of one or more sampling tips to the nasopharynx: device with single sampling tip (a) concept (b) schematic; device with multiple sampling tips (c) concept (d) schematic.

FIG. 5 depicts sampling tips contemplated by this disclosure (a) concept (b)-(d) example embodiments.

FIG. 6 depicts a device with a sampling tip in the form of an oblate spheroid (a) concept (b) representative embodiment.

FIG. 7 depicts a device with a cylindrical sampling tip (a) concept (b) representative embodiment.

FIG. 8 depicts a device with a cylindrical sampling tip (a) concept (b) (c) representative embodiments.

FIG. 9 depicts various configurations of tips contemplated for use with the devices disclosed herein.

FIG. 10 depicts various dispenser tips contemplated for use with the devices disclosed herein.

FIG. 11 depicts various medicament delivery mechanisms contemplated for use with the devices disclosed herein.

FIG. 12 depicts various configurations of nozzle tips and lumens contemplated for use with the devices disclosed herein.

### DETAILED DESCRIPTION

Disclosed herein are methods and devices for testing for the presence of viruses, including the SARS-CoV-2 Covid virus, using suitably designed transnasal or transoral probes. In some embodiments, the probe positions a sampling tip in proximity to the nasal pharynx.

In some embodiments, the method comprises the step of nasally directing a curvilinear or angled sampling tip to the nasal pharynx. In some embodiments, the method comprises the step of orally directing a curvilinear or angled sampling tip to the nasal pharynx.

Also disclosed herein are methods and devices for treating illnesses of the upper respiratory tract, and/or alleviating the symptoms of nasal illnesses.

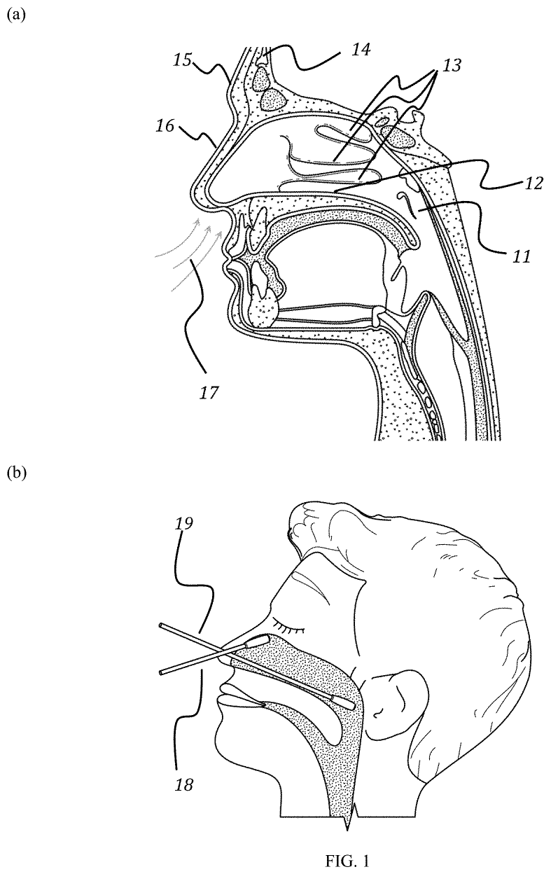

To aid in understanding the methods and devices of this disclosure, FIG. 1(a) shows the components of the head in and around the nasal cavity. The nasal pharynx 11, also termed the nasopharyngeal region connects the nasal cavity (above) to the oropharynx (below), behind the mouth. Other important features are the roof of the mouth 12, turbinates 13, skull bone 14, superior nasal cartilage 15, and nasal cartilage 16. Inhaled airflow is shown schematically as 17. It will be apparent from FIG. 1(b) that straight testing probes inserted through a naris are most easily directed upward to the roof of the nasal cavity, as shown in orientation 18. In order to reach the nasopharynx, horizontal insertion, with orientation 19, which is difficult to achieve, would be required.

The devices and methods disclosed herein use an alternate approach to current testing protocols, which require insertion of a probe via a naris. It will be apparent from consideration of the nasal cavity, shown in FIG. 1(a), that a nasally inserted probe with a downward bend or curve will be able to access the nasopharynx. Likewise, an orally inserted probe with an upward bend or curve will be able to access the nasopharynx.

Accordingly, disclosed herein are curved probes suitable for accessing the nasopharynx either transnasally or transorally. Also disclosed herein are methods for contacting the nasal pharynx, either by transnasal or transoral insertion.

As will be explained below, a curved probe will be able to contact a larger surface area of the nasopharynx than is possible with a linear probe. This property is beneficial for testing, since a sample derived from contact with a larger region of the nasopharynx will provide more sensitive and reliable data than from a single-point sample of the nasopharynx.

For transnasal testing, in order to increase yield and comfort an expansile tip which may be inflated and deflated with a balloon like mechanism has several advantages. Firstly the device can maintain a small size or diameter when passing through a naris, but with activation the sampling tip can expand in one or more directions and with any desired geometry including polygonal, oval, spherical, spheroid or other shape or form and with absorbent sample surface known to one skilled in the art covering all or part of the tip. Tip expansion and contraction may be repeated one or more times to ensure optimal sampling. The device can utilize a syringe-like mechanism involving air or fluid, or sponge, fiber, polymer, or like material which may be wetted and dedicated, and may be manually or automatically activated.

Further, manual activation in a naris itself would generate back pressure palpable to the individual performing the test, and incomplete activation would indicate to the practitioner that the tip was not placed in the nasal pharynx but remained intranasal.

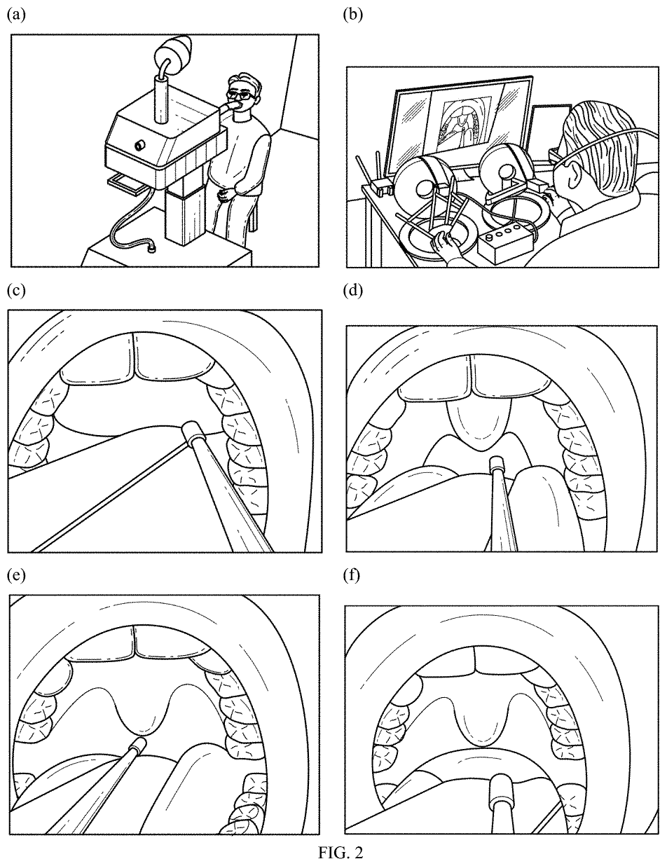

An existing procedure is shown in FIG. 2. The disclosed method uses a straight probe, (a swab or Q-TIP®) which is directed to the pharynx. Since the probe lacks an upward bend or curve, it cannot access the nasopharynx. Sensitivity is therefore decreased, and the procedure risks initiation of the gag reflex and potential gagging, coughing or vomiting.

The inventor notes that coronaviruses, and indeed other infectious pathologic agents, do not all behave like Streptococcus, and indeed the proper sampling for coronaviruses is in the nasal pharynx, and not the back of the throat. Inventor strongly believes that the current intranasal approach is suboptimal.

Clearly there is an unmet need to improve the comfort, accuracy, and sensitivity of testing for coronaviruses, including SARS-CoV-2, and other similar viruses.

Also disclosed herein is a method for treating or ameliorating the symptoms of headaches, nasal or other infections, irritations, inflammations, sinusitis or other sinus disorders, neuropathies, neuropathic, sympathetic mediated or other pain syndromes, common cold, flu, coronaviruses, for example Covid, or other viral infection, prion, protein, DNA, RNA mediated or related pathology or illness, autoimmune disorder or URI, the method comprising the step of applying a therapeutic agent, via a curvilinear or angled sampling tip, to the nasal pharynx.

A transoral approach has many advantages. Firstly, an altered geometry of the swab mechanism is disclosed in this invention. In its simplest form, the applicator attached to the sampling tip would be curvilinear or angled such that entering orally would allow for easy direction to the nasal pharynx for optimal coronavirus sampling. Or the entire apparatus could fit through an introducer, which could direct the applicator and/or the tip in an upward or cephalad direction to make contact with the nasopharyngeal area which is posterocephalic from the back of the throat. The introducer can be separate or integral to the applicator/tip apparatus.

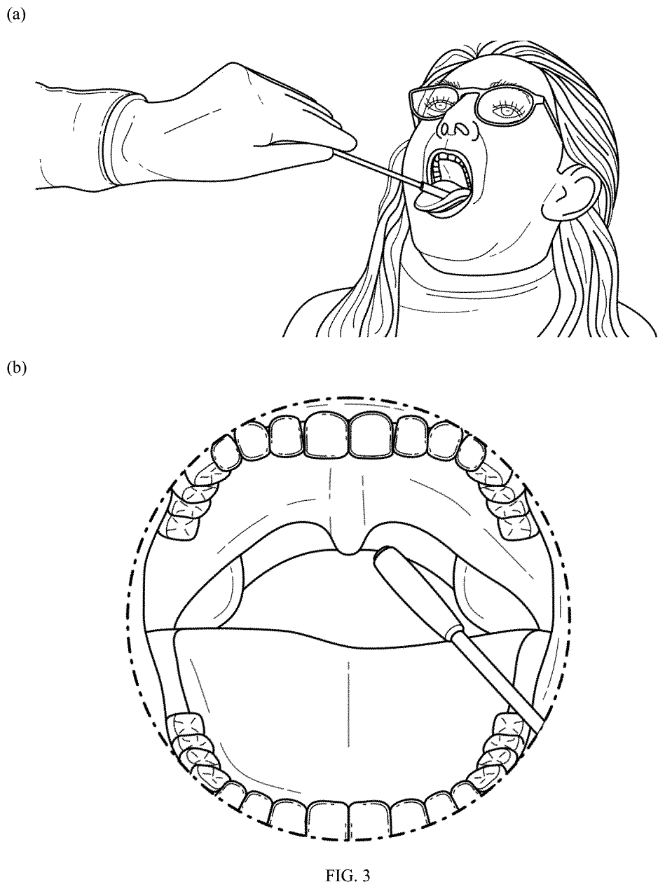

The introduction in this matter may be with the assistance of a tongue depressor type device, as depicted in FIG. 3, or with a bite block, or with the tested individual merely saying “ahh” or exhaling through the mouth.

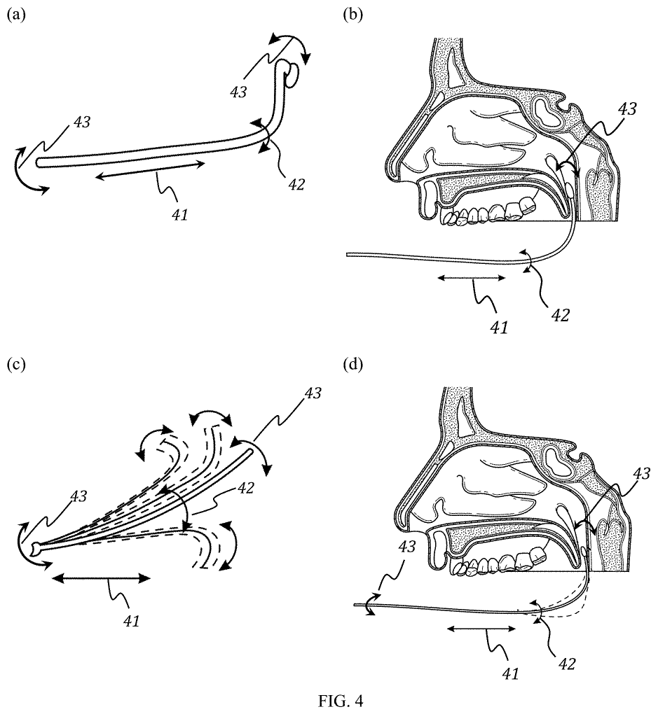

Approach of a curved or bent probe to the nasopharynx is depicted in FIG. 4. In FIG. 4(a) is shown a probe with a single tip. The curvature will orient the tip upward behind the oral cavity and to the nasopharynx. FIG. 4(b) shows the device oriented into the nasopharynx.

Some embodiments of the device further comprise one or more anchoring devices. The anchoring devices may consist of pads or plates to rest against the subject, thereby minimizing discomfort. Further embodiments may comprise mechanisms for adjusting the rotational, lateral, or vertical positioning of the tip relative to the one or more anchoring devices, thereby allowing precise manipulation of the tip for improvement of sampling and/or delivery of medicament.

A novel method for sampling would include the posterior portion of the sampling tip contacting the posterior nasopharynx and then the operator gently moving the entire device forward either via the introducer or by the applicator itself to make contact with the anterior portion of the nasal pharynx. This method would allow two samples to be obtained from two different areas in a single pass. Of course the operator can go back-and-forth a few times fairly quickly and atraumatically to increase viral particle uptake avoid false negatives.

Further, the operator can rotate the device to the right and to the left, posteriorly and/or anteriorly, to obtain samples from a wider section of the nasopharynx including loci more laterally. In some embodiments, the device comprises a shaft for insertion into the nasopharynx, and an applicator at the distal end oriented at an angle from the shaft. Possible manipulations of the device include forward and reverse lateral motion 41, left/right (windshield wiper) rotation about the shaft 42, and upward/downward inclination 43 (shown at both distal and proximal ends of the device). Manual rotation of the device around the axis of the shaft (indicated by the curved arrow surrounding the shaft of the devices in FIG. 4 will then sweep the applicator across a range of surface (indicated by the arcs at the distal end of the devices in FIG. 4, while maintaining the same orientation (with rotation) of the shaft. Hence twisting or rotating the device, introducer, or applicator will sample a greater surface area of the nasopharynx more completely. Therefore, not only does the curve in the probe allow access to the nasopharynx which is impossible with a straight shaft, but the curvature allows for a side-to-side (“windshield wiper”) motion which will sample a larger surface area than the same tip located on the end of a straight shaft.

A possible multi-sampling device is shown in FIG. 4(c). Each of the plurality of probes can contact the nasopharynx surface, providing a multiplicity of samples for testing. It will be appreciated that a multiplicity of samples will be likely to provide more robust diagnostic information than a single sample, from a single point of contact, which is scored “positive” or “negative”.

Using a similar design, with either a transnasal or a transoral approach such devices may be used therapeutically. They be introduced to spray a substance or compounds including but not limited to antivirals, anti-microbials, herbal, nutraceutical, anesthetic, vitamins, silver nitrate, copper, minerals, ions, oxygenating matrix, amino acids, proteins or other substances with medicinal value. Of course a distal saturatable tip can be introduced transorally with a hollow applicator comprising a proximal syringe, bulb, or other device to administer the compound to the absorbent tip.

Further applications to decrease pathogen density, including ultraviolet, infrared, ultrasound, infra sound, low-level electricity, or other energy can be utilized.

A fixed, expansible, or unfoldable probe can be employed to allow for greater surface contact.

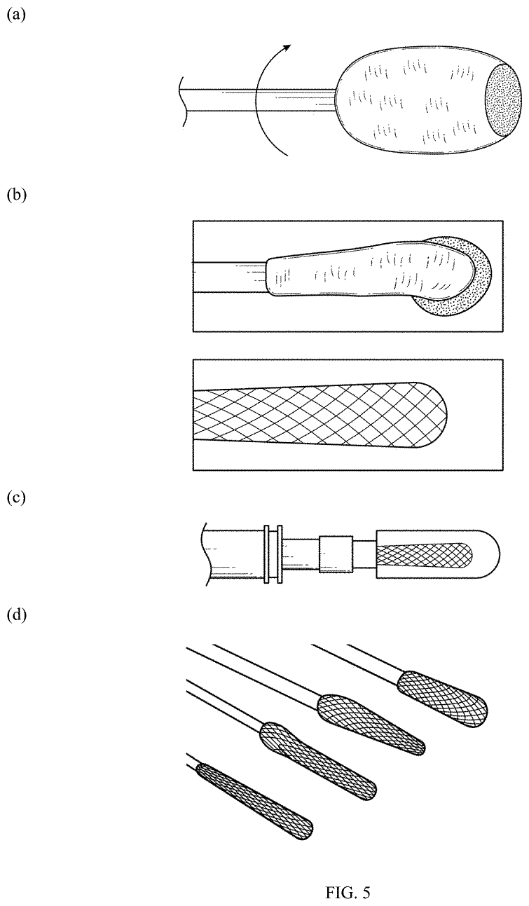

Additionally, the tip geometry can be polygonal, ovoid circular or any another geometry with the same, separate or different surfaces on each such that separate samples can be obtained if desired. Shown in FIG. 5 are several possible tip designs. Again the actual tip of a sampling device can be fiber, polymer, gum like, adhesive, hydrophilic or of another form or substance which will not break off and which will maintain integrity of the device structure. In its simplest sense, the device consists of a curved rod with a soft absorbent material in the form of a small sphere or ellipsoid at the distal end. The device may resemble a modified Q-TIP® like device would fit this description with an optimal length and with an optimal curvilinear or angled design not on a 180 or 0 degree geometry lengthwise. Additionally a polygonal, cylindrical, or other comfortable proximal grip device will increase operator comfort and improve sampling accuracy.

The distal end of any aforementioned diagnostic or therapeutic device may be fixed or moveable.

In one embodiment, the tip can rotate or move about a central shaft which may or may not be hollow and which may or may not serve as channel for moistening, agent delivery, or suction to, around or at the tip.

In another embodiment, the tip may rotate or move about one or more shafts at one or both ends which may or may not be hollow and which may or may not serve as channel for moistening, agent delivery, or suction to, around or at the tip.

In another embodiment, the tip may be a sphere, spheroid, ellipsoid, ovoid, polygonal or other form which may or may not rotate or move within a tubular or other construct allowing rotation or movement in a manner which may be linear, arcwise or otherwise. As a non limiting example for illustrative purposes, this may be in the general manner of construct of a deodorant or cosmetic roll on delivery device. The tip may or may not be expandable, compressible, or fixed in terms of volume or size. For maximum utility, the size differential between the diameter of the tubular or other construct containing the tip and the tip itself should be minimized to decrease trauma or unwieldiness of the device. The device may be of fixed shape, malleable, flexible or steerable by any modality known to one skilled in the art to optimize placement and use.

The tip may be utilized to turn, move or rotate to expose a different area of the tip when a to and fro sampling is done for one or more surface points, particularly a nasopharyngeal or oropharnyngeal mucosal or other surface point.

The tip may be utilized to roll or move in linear, arc like, or other manner during sampling maneuvers whereby it is moved or moves across or over more than a single surface point locus.

These maneuvers will increase the surface area sampled, and/or provide an increased sampling tip surface area exposure when a given surface is sampled.

When the device is to be used therapeutically, it may enter the nasal or oral areas and be directed to the desired location. A stabilizing comfortable anchoring anatomically appropriate design or component will maximize device stability and optimize placement during treatment. In some embodiments, this component is designed to rest gently on the face of the subject. By stabilizing the device, this component will reduce the effort required by the therapist to locate the device properly in the nasopharynx. In turn, the reduced effort can reduce therapist fatigue, and thus minimize the likelihood of improperly orienting the device, improving the subject's comfort during therapy.

When used therapeutically, the device may be placed in the appropriate area and the tip used to coat or “paint” the area one more time. This will engender greater mucosal uptake of agent and minimize dripping and loss of agent to other areas. Hence, agent delivery will be optimized as the agent is dosed sequentially giving time for the mucosa and submucosal structures to uptake the agent. This effect is enhanced particularly when using a rotating or otherwise non fixed application tip which presents a fresh or newly saturated or medicated tip section to the treated mucosal or other surface. The agent can be delivered to the tip by syringe like, constant, or varied pressure modalities, or by movement of portions of the tip into the agent reservoir.

In some embodiments, a therapeutic agent may be contained in a semi-solid substance at the tip. The semi-solid substance may be comparable in consistency to lip balm.

In some embodiments, a therapeutic agent may be formulated in a semi-fluid substance. The substance may have similar viscosity to cold cream or lotion.

In some embodiments, a substance comprising a therapeutic agent is adsorbed onto a material located at the tip. The material may be a natural or synthetic fabric, and may have a flocked texture so as better to contain the semi-solid substance. In some embodiments, the material is porous, so as to absorb the substance.

In some embodiments, a fluid or semi-fluid substance comprising a therapeutic agent may be contained in a reservoir. In some embodiments, the reservoir is located at the tip. In some embodiments, the reservoir is located external to the tip, and a tube is provided to deliver the substance to the tip. In some embodiments, flow of the substance from the reservoir to the tip is mechanically aided.

As in all devices mentioned herein, the tip may be solid, polymer, absorbent, smooth, variegated, punctate, spongelike, compressible, elastic, porous, nonporous, deformable, nondeformable or preformed or preshaped. The tip may be comprised of compact subfragments or filaments manufactured with natural, polymeric, or other materials.

When diagnostic devices are utilized, the tip may be broken off or pulled back into appropriate media or protective coating.

In certain embodiments, the device comprises a moveable part at the tip. In certain embodiments, the moveable part is a roller. In certain embodiments, the tip can be pushed back into media or to a storage position.

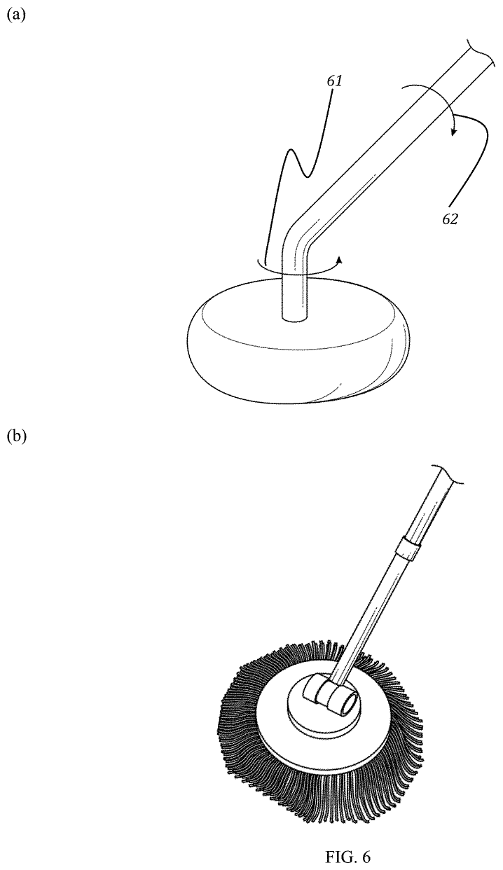

In some embodiments, the tip has the shape of a disk or oblate spheroid. The tip can then sweep across a significant surface area with minimal discomfort. The tip may optionally be mounted on a swivel. The design concept is depicted in FIG. 6(a); a possible embodiment of the tip is shown in FIG. 6(b). The device can be rotated about the shaft, shown with motion 61; in addition, the tip can freely rotate about its axis, shown with motion 62.

In some embodiments, the tip has the shape of a cylinder. The tip can then sweep across a significant surface area with minimal discomfort. The cylinder may optionally be mounted on a rotatable axis. The device can be rotated about the shaft, shown with motion 71; in addition, the tip can freely rotate about its axis, shown with motion 72.

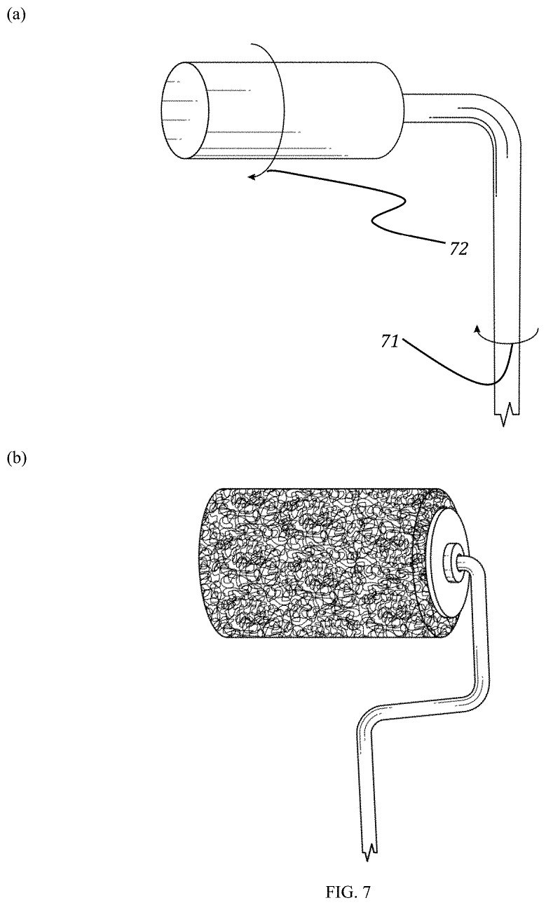

FIG. 7(a) depicts a design concept with a single support to the probe handle. A possible “paint roller” embodiment is depicted in FIG. 7(b).

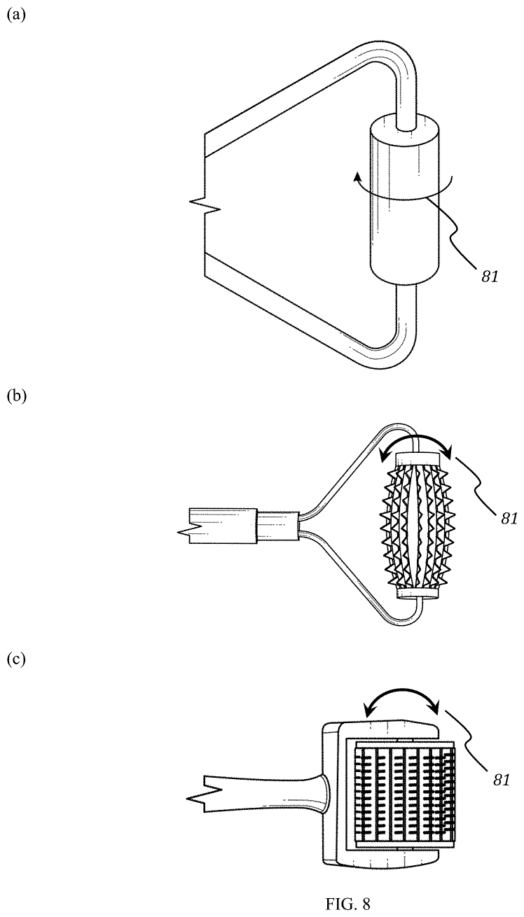

FIG. 8(a) depicts a design concept with a single support to the probe handle. Possible roller embodiments are depicted in FIGS. 8(b) and 8(c). Motion of the roller about its axis is indicated as 81.

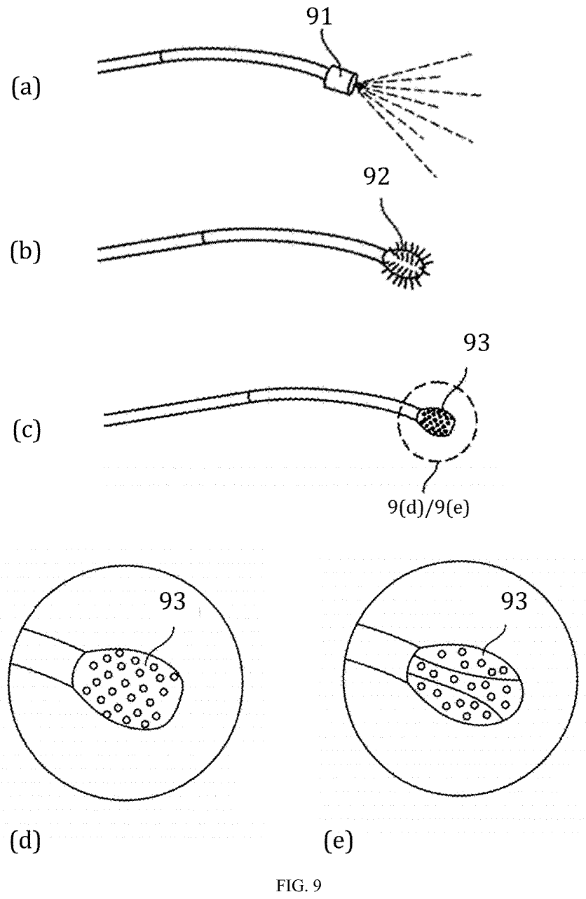

FIG. 9 depicts various configurations for tips intended for delivery of medicaments in the nasopharyngeal region. The device may be equipped with spray tip 91 in FIG. 9(a). Dispenser tip 92 in FIG. 9(b) may have a plurality of filaments, tubes or hair-like tentacles to optimize delivery of the medication by increasing the surface area that can be contacted and by controlling the amount of medication that flows through the dispenser. Alternatively, dispenser 93, shown in FIG. 9(c) may have a plurality of openings or perforations or be a porous or a semi-porous membrane. FIG. 9(d) shows an enlargement of one embodiment of the dispenser tip; alternatively, the dispenser tip can be divided into sectors, shown in FIG. 9(e), each of which can be fed by an individual canal in the shaft of the device.

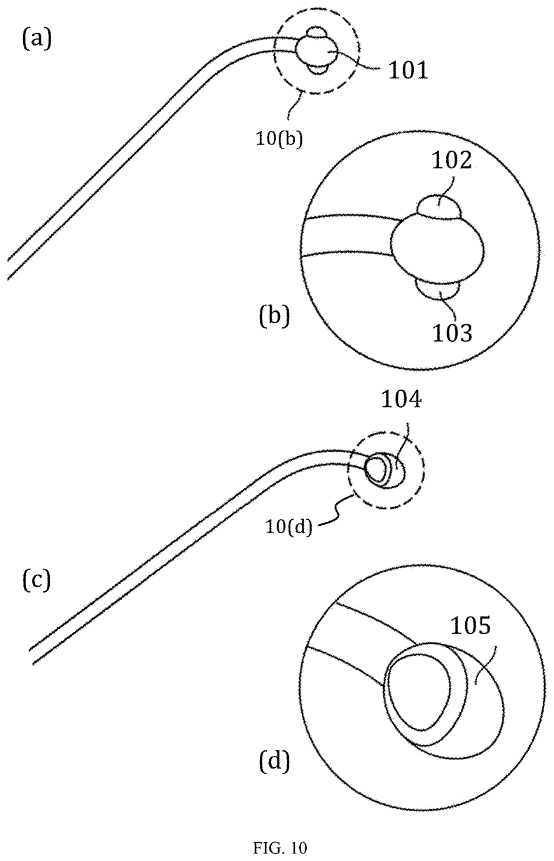

As shown in FIG. 10(a), the terminal end may be a dispenser 101 that may have a plurality of nodules or protrusions 102 and 102, shown in enlargement FIG. 10(b), that interface with a target area. The nodules may be permeable or configured in a manner similar to the embodiments described above such as in FIG. 9. The nodules are designed to optimize delivery of the medication by increasing the surface area that can be contacted and by controlling the amount of medication that flows through the dispenser. Also, the size and number of nodules may be varied along the surface area to control the amount of medicament applied to a desired area.

As shown in FIG. 10(c), the terminal end may be a dispenser 104 that may have a shape that resembles a Q-tip or be asymmetric in nature. Also, dispenser 104 may have a shape that is one-half, one-quarter of a Q-tip or otherwise shape-optimized. A Q-tip with one surface flattened 105 is shown in FIG. 10(d).

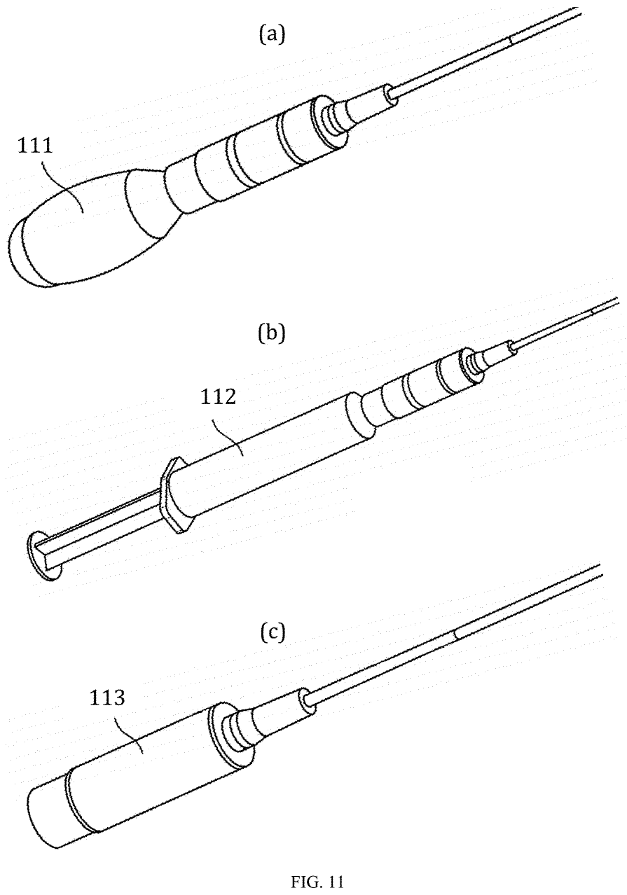

As shown in FIG. 11, a variety of medicament delivery mechanisms may be used to propel a liquid through the device, such as squeeze bulb 111, plunger 112, or pressurized container 113.

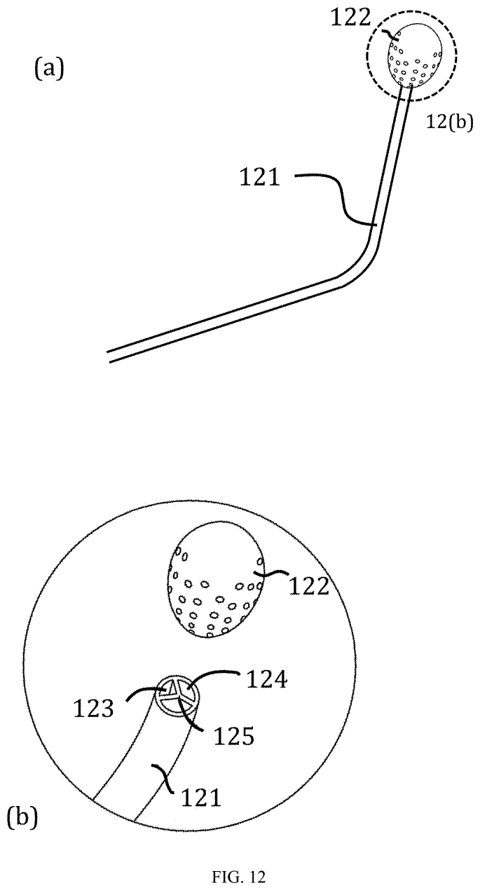

As shown in FIG. 12, the nozzle configurations shown above may be in the form of an elongated tube 121, connected to dispenser tip 122, that may be further configured to have a plurality of lumens 123-125 as shown in the enlargement of FIG. 12(b). The lumens may be uniform in cross-section or have different volumes to control the amount of medicament applied to a specific area. The lumens may also include filaments or other permeable materials as well as baffles and other types of inclusions therein to assist in directing the flow of a medicament to a predetermined area.

Similarly the device may be used to heat the area, rinse the area, or clean the area.

Lastly, intranasal or intraooral heat can be administered via a device with separate inhalation and exhalation cycles, and with moist heat, in order to decrease virus or micro blood. In the past, a device called the “Viralyzer” had limited success. It was noted that at 120 ° F., cold or flu viruses lyse. This would naturally shorten the duration and decrease symptoms of a cold or flu. The device had flaws in that it was essentially a hairdryer with no inhale or exhale phase.

Hence, a patient would be forced to breathe out against incoming hot air, which was suboptimal. Further hot dry air caused the mucosal surfaces to dry and crack. This was uncomfortable, and could in fact lead to increased viral colonization within these cracks. Hence moist heat delivered only during inhalation, through one-way or other valves known to one skilled in the art, such as those found on anesthesia breathing circuits or scuba diving equipment, would increase the efficacy of this device and decrease its side effects. Next, including an infrared or other surface temperature monitor which can be situated integral or separate from the device and which was coupled with the device, would prevent burns by decreasing heat production which would vary inversely with mucosal surface temperature.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed is:

1. A device for contacting the nasal pharynx via either the nasal cavity or the oral cavity, the device comprising:
a rodlike shaft for manual manipulation; and
one or more sampling tips at the distal end;
wherein the device has an upward curve or bend, providing access to the nasal pharynx.

2. The device of claim 1, wherein the one or more sampling tips comprise absorbent material.

3. The device of claim 2, wherein the one or more sampling tips are substantially spherical in shape.

4. The device of claim 2, wherein the one or more sampling tips are substantially ellipsoidal in shape.

5. The device of claim 4, wherein the one or more sampling tips have the shape of a prolate ellipsoid.

6. The device of claim 4, wherein the one or more sampling tips have the shape of an oblate ellipsoid.

7. The device of claim 2, wherein the one or more sampling tips are substantially cylindrical in shape.

8. The device of claim 2 wherein each of the one or more sampling tips is connected to the rodlike shaft via a single point of attachment oriented on one end of its symmetry axis.

9. The device of claim 2, wherein each of the one or more sampling tips is connected to the rodlike shaft via two points of attachment oriented on opposite ends of its symmetry axis.

10. The device of claim 2, wherein the one or more sampling tips can freely rotate on an axis.

11. The device of claim 10, further comprising one or more reservoirs in proximity to the one or more sampling tips.

12. The device of claim 11, wherein the rotation of a sampling tip on an axis causes distribution of material within a reservoir onto the surface of the tip.

13. A method for detecting or quantifying a microbe in the upper respiratory tract; the method comprising the steps of:
transnasally or transorally inserting a device as recited in claim 1;
contacting the tip of the device with the nasal pharynx;
manipulating the device so as to harvest material from the nasal pharynx; and
withdrawing the device.

14. The method of claim 13, wherein the microbe is a virus.

15. The method of claim 14, wherein the virus is a coronavirus.

16. The method of claim 14, wherein the virus is SARS-CoV-2.

17. A method for treating or ameliorating an ailment of the upper respiratory tract; the method comprising the steps of:
transnasally or transorally inserting a device as recited in claim 1;
contacting the tip of the device with the nasal pharynx;
manipulating the device so as to apply medicament contained in the one or more reservoirs onto the surface of nasal pharynx; and
withdrawing the device.

18. The method of claim 17, wherein the medicament is an antibacterial.

19. The method of claim 17, wherein the medicament is an antiviral.

20. The method of claim 13, wherein the device is inserted transnasally.

21. The method of claim 13, wherein the device is inserted transorally.
